(19) United States
(12) Patent Application Publication
(10) Pub. No.: US 2022/0008006 A1
(43) Pub. Date: Jan. 13, 2022

(54) System and Method for Identifying and Tracking Infected Individuals

(71) Applicants: Ian Paul Shadforth, Morrisville, NC (US); Andrew Hugh Dubberly, Palo Alto, CA (US)

(72) Inventors: Ian Paul Shadforth, Morrisville, NC (US); Andrew Hugh Dubberly, Palo Alto, CA (US)

(21) Appl. No.: 17/226,345

(22) Filed: Apr. 9, 2021

Related U.S. Application Data
(60) Provisional application No. 63/008,181, filed on Apr. 10, 2020.

(51) Int. Cl. A61B 5/00; A61B 5/024; A61B 5/11; A61B 5/01; G16H 50/30; G16H 50/80

(52) U.S. Cl. CPC A61B 5/6801; A61B 5/024; A61B 5/746; A61B 5/688; G16H 50/80; A61B 5/0015; A61B 5/11; A61B 5/01; G16H 50/30; A61B 5/0004

(57) ABSTRACT

Briefly, a human-wearable sensor package is provided that can be conveniently worn by an individual. The sensor package contains multiple sensors that can take physiological measurements that when evaluated may indicate that the individual has contracted an infections disease. In response, the sensor package may generate a local alert to the individual, or communicate an alert message to remote receivers, such as to other people close to the infected individual or to remote health care providers or services. In another example, some of the sensors may be located remotely from the individual.

### RELATED APPLICATIONS

This application claims priority to U.S. provisional application No. 63/008,181, which was filed Apr. 10, 2020 and entitled “Method for Identifying and Tracking Contact with Individuals Showing Markers of Infection During an Epidemic” which is incorporated herein.

### FIELD OF THE INVENTION

The field of the present invention is the collection of physiological data from humans and using that data to algorithmically assist in tracking and slowing the spread of infectious disease.

### BACKGROUND

The novel coronavirus 19 unleashed its horror on the world in December 2019, and its effects and devastating impact on the world will be felt for years to come, both for its human and economic tolls. The novel coronavirus 19 is considered to be 2 to 3 times more contagious than the common flu, and the resulting Covid 19 disease can be up to 15 to 20 times more deadly than the seasonal flu. With such high infection rates, frightening mortality, and easy paths to worldwide spread, the World Health Organization and many countries announced Covid 19 was a full-blown pandemic by early 2020. In response, countries virtually shut down and required citizens to shelter in place. Despite global precautions and fast reactions, the number of infections reach into the millions, and the number of deaths into the hundreds of thousands, and death tolls continue to rise as of today. Not only is Covid 19 causing untold human tragedy, it is wreaking havoc and overwhelming hospital systems throughout the world. Further, even if and when this pandemic is brought under control, its economic impacts may last decades, as world economies reel from the loss of trillions of dollars in value, and the evaporation of tens of millions of jobs.

It is well understood that the best way to manage a pandemic is to catch the outbreak early, and aggressively contain the spread to a localized area. However, as with Covid 19, such a response was not possible due to the inability for countries to test their populations and understand the extent and speed of virus transmission. This was especially difficult for Covid 19, which not only had a rather unpredictable and long incubation period of between 2 and 14 days, but also could be spread by individuals that had the virus but had no or only minor symptoms. These asymptomatic spreaders could infect a wide swath in any public area or gathering and be completely unaware of the consequences of their innocent actions.

It took months for countries to develop and deploy accurate testing kits that could confidently enable public health organizations to make meaningful and correct decisions regarding quarantines and public shutdowns. Those months, quite literally, caused tens of thousands if not hundreds of thousands of lost lives and untold suffering for individuals and families.

It is also widely believed that the world is likely to see more and more severe pandemic outbreaks as populations grow, the climate changes, and clean water and sanitation systems become stressed. Accordingly, it is the obligation of the scientists, thinkers, and engineers of today to put in place those public health systems and processes that can stop pandemic in its tracks, or at a minimum relieve the human and economic horror by assisting public health officials and individuals in defining the most efficient and effective attack against the virus spread.

Further, although the world's attention is currently on viruses due to Covid-19, other microbes such as bacteria, fungi and parasites can be equally, if not more destructive to human life and economies. Accordingly, although the focus here is on virus, it will be understood that these same of very similar concepts extend to bacteria, fungi and parasites.

We might agree that “systems” are sets of related elements that act together in a way that has some meaning—or in Ross Ashby's terms, a list of variables that map to a single value transformation. Living things are systems that can recreate themselves. Maturana and Varela called this process “autopoiesis”—self-making. A virus is a living thing (just barely)—a system that recreates itself. The animals (including humans), on which viruses depend, are systems, too.

In addition to recreating themselves, living systems also maintain certain internal and external relationships. That is, they maintain “dynamic equilibrium” for quite a number of variables. Equilibrium suggests “balance” or “stability”. Dynamic equilibrium suggests a “flow” of a “stock” through a system, where the “in-flow” equals the “out-flow”, as water moves through a lake or a bathtub, while its level remains constant. (For more on “dynamic equilibrium”, see Donella Meadows' book “Thinking in Systems”.)

Mammals maintain dynamic equilibrium for many variables, (e.g. temperature, blood pressure, water, oxygen, blood-sugar, salt, and many more minerals). Maintaining a set of variables in dynamic equilibrium is called “homeostasis”. In the process of reproducing itself, the Covid-19 virus disrupts human homeostasis, in particular the process of maintaining oxygen levels, for example. A disruption of homeostasis is a way that viruses and bacteria often kill people. The processes of self-maintenance are many and richly interconnected. They also change with age, which correlates with death rates varying with age.

Homeostasis is made possible by a process of “self-regulation” or “control”. “Control” is a technical term from systems theory (i.e., “cybernetics”). To control is to “regulate” or “manage” or “maintain” a variable so that it stays below a threshold (e.g., temp <72° F.) or within a range (e.g., 70 mg/dL<BG<140 mg/dL). A control system requires apparatus for measuring (sensors) and acting (actuators). It also requires a way of comparing a “goal” (desired value) with what's measured (current value) and if there is a difference (an error or delta), then switching on the necessary compensatory action. This process involves the flow of information (not a stock) through a system, into an environment, and back—that is, feedback.

In machines, a thermostat+furnace+air-in-the-room is an example. The thermostat+furnance “control” (or regulate) the temperature (significant variable) of the-air-in-the-room. Your body uses quite similar processes to regulate temperature, blood pressure, blood sugar etc.

When being disrupted by an outside force, a system can maintain control only up to a point. If the outdoor temperature drops to −100, your furnace may not be able to keep up. Likewise, if you binge on carbs, your blood-sugar level will spike; if you do that repeatedly for a long time, you may overwhelm the system and have full-blown diabetes. The ability of a system to withstand a disturbance is called “variety”. (Ross Ashby describes variety as a measure of information.) A system that is “out-of-control” either explodes (as the virus is doing), or it oscillates wildly (as the stock-market is doing). The relationship between viruses and hosts is a larger system—a “system of systems”, or an “ecology”.

Humans are systems of systems. (Your body is an ecology.) Of course, we also live within a natural ecology. And on top of it, we have built and now live in vast social-technical ecologies, comprised of a growing array of social-technical systems (STS)—for example, languages, the airline system, the healthcare system, local, state, and national governments, the World Health Organization, and many more. All of these ecologies, at each level, involve interactions which create disturbances, which the systems attempt to “control” so as to maintain their manner of living.

Social-technical systems can “control” viruses and their spread. However, they may not have enough “variety”—and as the virus stock expands, it can overwhelm the control systems. Requisite variety is what's needed to counter likely disturbances. Of course, “likely” is a probability of an event occurring over a period of time. Picking an acceptable likelihood and period is a systems design choice. There are trade-offs. More variety affords greater protection, but more variety also requires more resources. Simply put, variety is expensive. In the end, the amount of variety included is a choice, a matter of value and values.

In the absence of an informative measure (from a sensor) there can be no appropriate actuation and hence the system cannot be effectively controlled. In the case of COVID-19 spread, rate of infection is the dependent variable, and restriction of contact between those infected and those not infected is one means of actuation. However, there is not an effective sensor as testing invariably comes too late in the cycle to prevent many contacts between infected and non-infected. In this case, the variety of the system (society) in response to the introduction of COVID-19 may not be being explored as the system progresses immediately to “out of control”.

The Israeli government has been public and aggressive about using phone data (location), which gives potential contacts, as a way of tracking networks, which is a way of tracking possible infections. Various other applications have also been developed that can use GPS data from cell phones to track population measures of social distancing, such as the Unacast Social Distancing Scoreboard (https://www.unacast.com/covid19/social-distancing-scoreboard).

In Singapore, an app called TraceTogether uses Bluetooth signals exchanged between cell phone to produce lists of contacts between people using the app (https://www.mobihealthnews.com/news/asia-pacific/singapore-government-launches-new-app-contact-tracing-comb at-spread-covid-19).

The digital, connected, Kinsa thermometer has been shown previously to provide better tracking of the spread of flu, at the population level, than the CDC. Currently (March 2020), this data is likely to also be tracking the spread of COVID-19 in the USA (https://www.nytimes.com/2020/03/18/health/coronavirus-fever-thermometers.html).

Although these are all very positive steps towards tracking the potential spread of infection, many of them operate only at the population level—providing an estimate of the spread of infection. The TraceTogether app provides interpersonal contacts, which work at the individual level, but only if every individual has the application on their phone and operational (i.e. open, with Bluetooth turned on). Also, TraceTogether provides no early warning that an individual may be infected, but allows contacts to be traced once infection is confirmed, causing a significant lag between contact and warning. The Kinsa thermometer would help provide an early warning, but does not provide contact tracking. It is also reliant on the user of the thermometer to regularly check their temperature, ideally before they think they may have a fever.

### SUMMARY

Briefly, a human-wearable sensor package is provided that can be conveniently worn by an individual. The sensor package contains multiple sensors that can take physiological measurements that when evaluated may indicate that the individual has contracted an infections disease. In response, the sensor package may generate a local alert to the individual, or communicate an alert message to remote receivers, such as to other people close to the infected individual or to remote health care providers or services. In another example, some of the sensors may be located remotely from the individual.

### BRIEF DESCRIPTION OF THE DRAWINGS

FIG. 1 is an illustration of a human wearing a sensor package in accordance with an embodiment of the present invention.

FIG. 2 is a functional block diagram of a sensor package in accordance with an embodiment of the present invention.

FIG. 3 is a block diagram of a method for using a sensor package in accordance with an embodiment of the present invention.

FIG. 4 is a block diagram of a method for using a sensor package in accordance with an embodiment of the present invention.

FIG. 5 is a functional block diagram of a distributed sensor package in accordance with an embodiment of the present invention.

### A BRIEF DESCRIPTION OF ONE EMBODIMENT OF THE INVENTION

We present here embodiments that mitigates these issues, which will generally be described with reference to the present Covid 19 pandemic outbreak. However, it will be understood that these embodiments can be more widely applied and deployed depending upon public health requirements. For example, these embodiments are generally described as they relate to the spread of a viral infectious disease, but are also applicable to bacteria, fungi, parasites, and diseases spread by parasites. In this example the system consists of a number of human physiological sensors, the data from which are interpreted by an algorithm to provide early warning of potential infection. It will be understood that environmental and location sensors may also be used. This warning may be immediately available to the user. It may also be shared with individuals with whom the user interacts to provide a pre-emptive means by which individuals may start to self-isolate before the disease diagnosis is available. Also, the information may be shared with other relevant individuals or entities, such as the individual's healthcare provider or a regional healthcare facility. Further, such information, provided it is properly protected for privacy purposes, may be used by regional, national, or worldwide health organizations in implementing effective public health policies regarding an emerging virus, bacterial, fungal, or other threat. The following text, along with accompanying figures, provides a description of one embodiment of this invention. Other embodiments of the invention may be apparent to those skilled in the art, a few examples of which are subsequently presented. However, it will be understood that the devices, sensors, algorithms, and systems described herein may be widely applicable to monitoring and controlling various public health problems, such as localized epidemics and widespread pandemics.

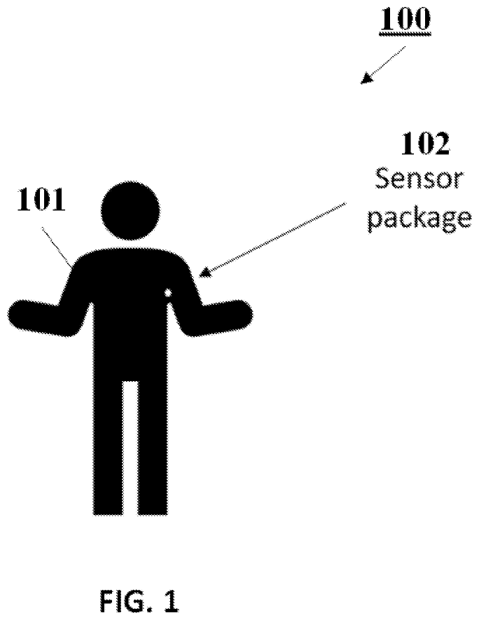

For a particular threat of interest, a sensor or a set of sensors in a package are designed to monitor one or more biometrics of an individual that may help provide an indication of whether or not the individual is currently infected and whether the current individual has been infected and is now in recovery. It will be understood that different pandemic threats would require different sensors or sensor packages. A sensor package is to be worn by members of a population, with it being highly desirable for the sensor package to be worn by a significant portion of the population to assure complete and accurate data. FIG. 1 shows an individual sensor system 100 with a human 101 wearing a sensor package 102. The human 101 is illustrated with the sensor package 102 in one of several suitable locations for the sensing package, the armpit. It will be understood that such a sensor or sensor package may be installed in various places on or in the human body The sensing package is small, unobtrusive and may be worn continuously for long periods of time without causing discomfort. In one embodiment, the sensor may be implanted. In other cases the sensor may be adhesively fixed to the human 101, or may be frictionally held using clothing or banding. The power demands of the sensor are such that infrequent re-charging is needed through the use of low-power electronics and potential energy harvesting technologies. In one embodiment, the recharge period is every three months. In another example, the sensor or sensor package is powered by a battery such as a coin battery, or has the capability to harvest power from light, motion, or chemical energy. It will be understood that a sensor or sensor package can be powered in a number of different ways, depending upon the particular application and installation location.

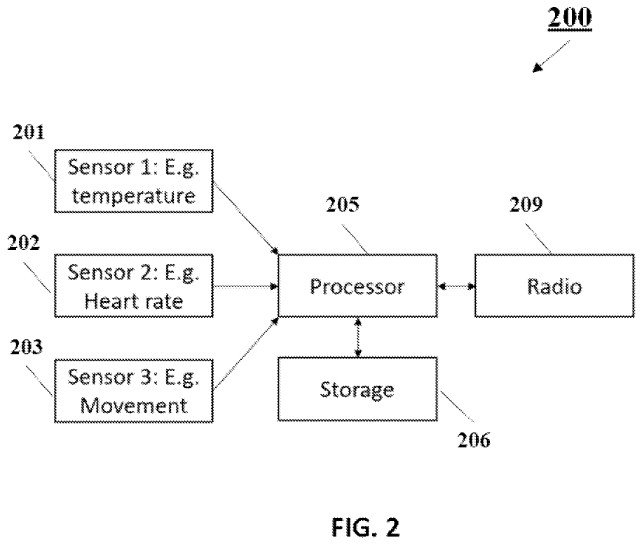

Referring now to FIG. 2, the sensing package 200 comprises a number of sensors (201, 202, 203), a processor 205, data storage 206 and communications chipset 209. In one embodiment, which would be applicable for a system designed to monitor for Covid 19, the sensors are:

a) Resting heart rate monitoring 202;

b) Resting temperature monitoring 201; and

c) Continuous activity monitoring 203.

It has been found that a variation of these three measurements can provide a good indication that an individual has contracted an infectious disease. It will be understood that other sensors may be used in the sensor package according to application specific requirements. The sensor package may communicate with the user's portable device, such as a smartphone. Accordingly, some of the measurements, for example movement, may use the accelerometer in the smartphone, or may use it to supplement data collected from the local package sensor. 203. Also, it is desirable to have as much of the sensing as possible be done by the sensor package 200, as often the user will be too far away from the sensor for immediate communication, or to provide meaningful localized data. However, the radio 209 may be able to determine that the smartphone is nearby, and the system may thereby be able to use the position location information from the smartphone as a meaningful proxy for location of the user.

For e example, resting heart rate for an individual increases when an individual is infected with a virus (https://journals.plos.org/plosbiology/article?id=10.1371/journal.pbio.2001402)

Temperature (ideally resting to distinguish from exercise) increases when an individual is infected with certain viruses. A means to identify that the individual is at rest is required, hence the activity monitoring component. There may also be changes in activity characteristics, such as lower gross movements when infected (staying in bed, at home, moving more slowly), or, at a finer resolution, shivering. It will be understood that other sensors may be useful for more confident and accurate detection of an infection or recovery. However, it will be well understood that there is a trade-off between the number and accuracy of the sensors, and the ability to keep the cost and size such that the sensor can be readily and inexpensively deployed throughout a population. Thus, it will be understood there is a system wide trade-off in the technology that encourages lower cost, lower size, more comfort, and longer life, such that solid information can be obtained from a larger portion of the population over a longer period of time, thus enabling the most effective public health response. In other cases, for example where the individual desires more accurate information, additional sensors and more accurate sensors may be used. Accordingly, the system may have different types of sensors depending upon an individual's or areas specific health needs, with all of these various sensor readings feeding into a common algorithmic process.

Technology: Processing could be maintained locally (at the edge) with outputs from the sensing package only required when the appropriate conditions are met. This helps to conserve battery life for true continuous monitoring. Further, such local control also helps maintain privacy of personal health data.

Communications: Offload of an alert could be accomplished using BLE (Bluetooth low energy) when necessary through broadcast and is thus not just limited to an individual's own phone, but any BLE-capable device could act as the conduit for the alert. Inter-sensing package communication may also be achieved using BLE.

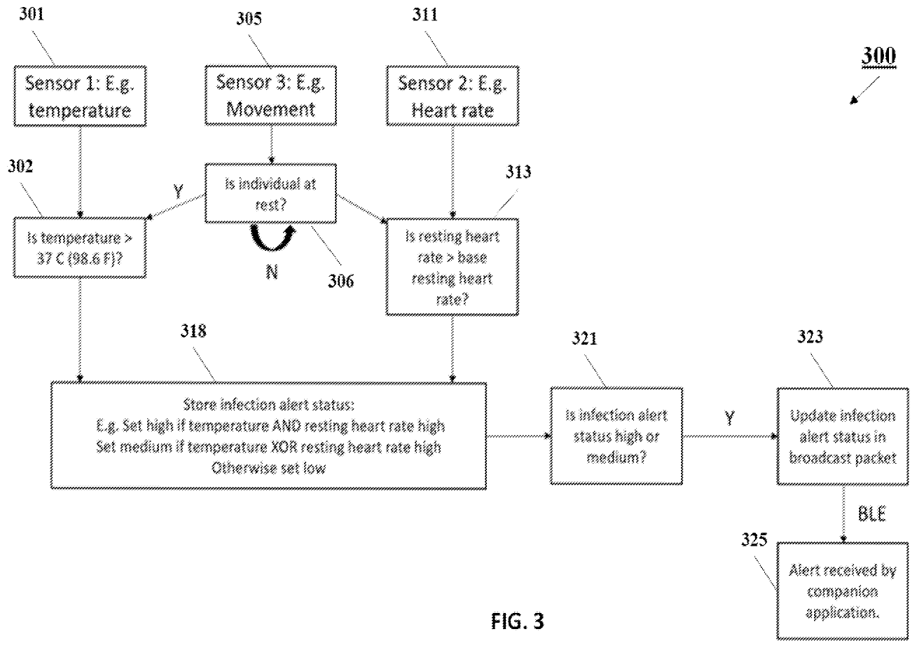

The processing flow 300 of this embodiment of the invention is disclosed in FIG. 3. As shown in FIG. 3, three sensors are associated or attached to a human user. Sensor 301 is able to measure the temperature of the user, sensor 305 is able to measure movement of the user, and sensor 311 is able to measure the heart rate of the user. The sensor 305 detects movement, and then the processor may determine if the individual is at rest as illustrated in block 306. Being at rest may be set for longer or shorter periods of time depending upon the type of user, the age of user, or other user or environment specific information. Determining whether the individual is at rest may also be different depending upon whether the temperature is being measured 301 or the heart rate is being measured 311. If it is determined in block 306 that the individual is at rest, then the temperature may be compared to a normal temperature as shown in block 302. Whether or not this temperature is high or low may then be reported to an information and alert process 318. In similar manner, if the individual is at rest for purposes of heart rate as shown in block 306, then as illustrated in block 313 that heart rate may be compared to a base heart rate. In a similar manner whether or not the heart rate is too high or too low can then be reported to block 318. For purposes of taking the resting heart rate, it will be appreciated that the base heart rate for the individual must be known. Accordingly, the sensor will use historical data from the user to determine a base heart rate. Alternatively, absolute high and low threshold may be set. Although heart rate is being discussed relative to an abnormally high heart rate, it will be appreciated that some infectious diseases may cause the heart rate to be abnormally low

In block 318, the system 300 determines whether or not there is a high alert status or a moderate alert status. It will be appreciated that other levels of alert may be determined. For the high alert status, this may be set depending upon if both the temperature and resting heart rate are too high. In another example, the high alert may be set if the temperature sensor alone is critically high, or if the base rate reading is critically high. In this way, it may not be a simple passing of a threshold that determines an alert status, but there may be a comparison between the measurements made at the different sensors. In another example, a medium alert may be set if there is reason for caution, for example, if the heart rate is moderately high and the temperature is normal, or the temperature is moderately high and the resting heart rate is normal.

If block 318 determines that an alert condition exists, that alert condition can be passed to block 321, which determines the importance of the alert. In block 321, the system may determine whether or not that the alert is significant enough to broadcast to health officials or medical personnel or is not critical and can just provide a local alert to the user or to the user's smartphone. In block 323 the actual communication or broadcast package is assembled, and if need be, the package is transferred via a wireless radio to an alert companion application as illustrated in block 325. In one example this alert companion application may be in the user's smartphone, or in some cases may be another radio enabled device.

To protect privacy, the system 300 does not broadcasting continuously, i.e. but only when an alert condition is present due to potential infection, which resolves a potential for unwanted tracking or other concerns. It will also be understood that the individual may be allowed to have some control over the dissemination of health information, for example setting some level of confidence indicator prior to allowing personal information to be shared into the public networks. The location of the sensor is also fairly discrete. The sensing package would thus provide a means to identify individuals who are showing symptoms before they are aware that they are potentially infected. The system would identify candidates for further diagnostic testing. Additionally, this information can be used to alert an individual that they have had a likely exposure, and therefore they are on notice that they may become an asymptomatic spreader. With this information, the individual may take the additional step of seeking a blood test to confirm whether or not they have the virus, enabling them to make the critical decision to quarantine or avoid human contact until the virus is no longer active in their system.

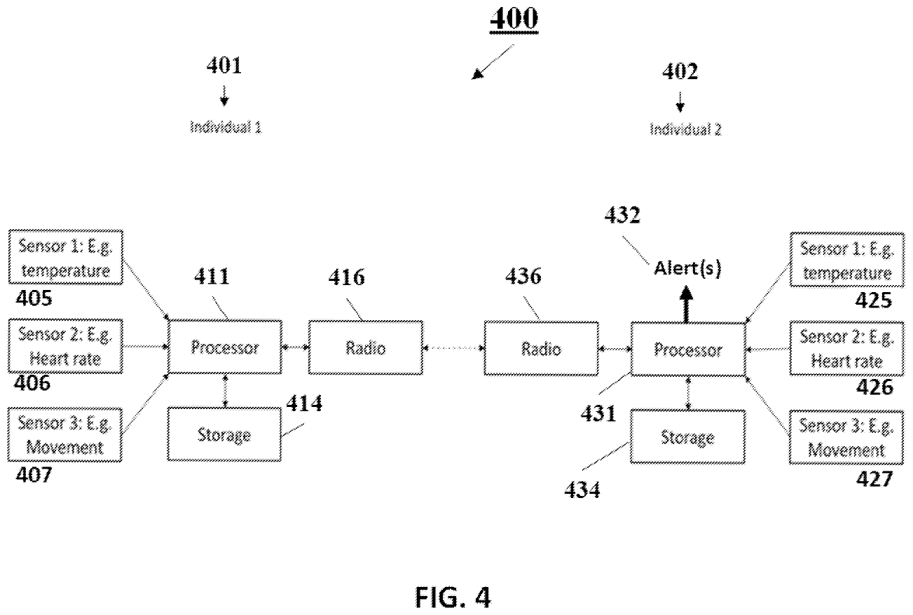

In an extension to this, the use of periodic (e.g. every 1 min) BLE broadcasts would be stored temporarily by other individual's sensing packages which would allow for a reconstruction of close contacts. This multi-person scenario 400 is depicted in FIG. 4. System 400 is illustrated with two individuals, individual 401 and individual 402, which are in close proximity to each other. In some cases, close proximity can mean within 10 feet, although other distances may be used depending upon the type of radio or sensors being used. Individual 401 has a sensor packet which contains movement sensor 407, heart rate sensor 406, and temperature sensor 405. These three sensors report to a processor 411, which then stores information in a local storage area 414. As described with reference to FIG. 3, individual 401's system will determine if there is a risk that 401 is currently infected, and if so, will broadcast a localized message from radio 416. That message may be received by radio 436, which is associated with individual 402. Information that individual 401 is potentially infected is then stored by processor 431 in storage area 434. If radio 416 is set to broadcast an alert every one minute, then individual 402 will also be aware of how many minutes they were in close proximity to individual 401. In this way, individual 402 may make a determination to seek medical help, be tested, or quarantine. Further, this information may be used by some central data collection process for conducting contact tracing using a public health service organization.

In a similar manner, individual 402 has a sensor packet which contains movement sensor 427, heart rate sensor 426, and temperature sensor 425. These three sensors report to a processor 441, which then stores information in a local storage area 434. As described with reference to FIG. 3, individual 402's system will determine if there is a risk that individual 402 is currently infected, and if so, will broadcast a localized message from radio 436. That message may be received by radio 416, which is associated with individual 401. Information that individual 402 is potentially infected is then stored by processor 411 in storage area 414. If radio 436 is set to broadcast an alert every one minute, then individual 401 will also be aware of how many minutes they were in close proximity to individual 402. In this way, individual 401 may make a determination to seek medical help, be tested, or quarantine. Further, this information may be used by some central data collection process for conducting contact tracing using a public health service organization.

Further, after individual 402 receives a message that a nearby user, individual 401, may be infected, processor 431 may issue an alert 432. This alert may be, for example, a vibration, an audible sound, or a local visible message on the smartphone. It may also include alert messages sent to other nearby devices, and even alert messages to a centralized healthcare service. If individual 402 receives multiple spaced-apart messages from individual 401, then individual 402 may be able to assess how long individual 401 has been nearby. For example, the system may be set such that individual 402 may be near an infected individual for a maximum of three minutes before an alarm goes off. In other cases, the amount of time that individual 402 may have been exposed is stored and then used later to assess what type of testing or medical help individual 402 should seek.

While it may be possible with sufficient warning to have substantial parts of the population outfitted with sensors, and participating in the system wide processes, in many cases such warning may not exist. In these cases, rather than attempt to do a population-wide or regional-wide protection system, it may be more effective to deploy the sensors in what might be called “beachhead” populations. For example, it may be far more practical to have all healthcare workers in a city outfitted with the sensor and thus be able to monitor and make decisions regarding this high priority group. Other groups, such as the National Guard, Army, police, first responders, firearm men, could also be outfitted to assure that these high risk individuals are protected, and enable public healthcare officials to deploy resources with better and more complete information.

Resting periods are determined to be the period in which cumulative motion over a 10-minute bin, as continuously measured using a 9-axis accelerometer, are least for a period of 24 hours. In this way, a daily measurement of resting metrics is ensured, with more frequent assessments if the individual becomes less active, which may occur if they start to feel ill. It will be appreciated that other criteria may be used according to user, application and environment specific requirements.

A fever is medically defined as any body temperature above the normal of 98.6 F (37 C). In practice a person is usually not considered to have a significant fever until the temperature is above 100.4 F (38 C). This embodiment would provide an alert when resting body temperature rises above 98.6 F (37 C). It will be appreciated that other criteria may be used according to user, application and environment specific requirements.

Resting heart rate is measured as the average heart rate over the 10 minute bin identified as an “at rest” bin by the assessment of the accelerometer data. An “elevated resting heart rate” is defined as an increase in resting heart rate of 10% above the individual's baseline resting heart rate. The “baseline resting heart rate” is defined as the median resting heart rate measured over the previous 30 days, excluding measurements identified as “elevated resting heart rate”. It will be appreciated that other criteria may be used according to user, application and environment specific requirements.

An alert status of “high” is set if both the resting temperature AND resting heart rate are elevated. An alert status of “medium” is set if either one of resting temperature or resting heart rate is elevated. A null alert status is set if neither condition is met. It will be appreciated that other criteria may be used according to user, application and environment specific requirements.

The alert status is communicated to the individual via a BLE connection to a companion application on a connected device, such as their smartphone. The alert status is anonymously shared with the sending packages of other individuals so they can be aware of their contact with a potentially infected individual.

The selected set of sensors is selected to enable an individual to establish a baseline of biometric measurements prior to any infection. In this way, ideally the individual would begin wearing the sensor well before any substantial risk of infection exists, thereby enabling a confident and accurate baseline to that individual's biometrics to be established. Then, if the individual does become infected, the changes in one or more biometrics can lead to a more confident prediction that the individual has contracted the virus, and may even be able to provide an indication of how severe and how fast the infection is affecting that particular individual. These individual pieces of information, are of course critical to the individual and the healthcare support around that individual, but are also incredibly important to enable public healthcare officials to make reasoned and effective decisions.

### OTHER EMBODIMENTS

While we have presented above one example embodiment, other embodiments and alternative implementations will be apparent to those skilled in the arts. Without limitation, such other embodiments may include:
a) The use of multiple, spatially separated, sensors connected to one or more centralized processing units. For example, but without limitation, this may include a body temperature sensor in the individual's armpit, a motion sensor and heart rate sensor on their wrist (for example in a smartwatch), all of which are connected to a processing and storage unit in an individual's phone, which may provide for further connectivity to remote processing and storage units in the cloud.
b) The use of a combination of sensors on the individual's body with sensors not on the individual's body. For example, but without limitation, this may include a heart rate sensor on the body, with a movement sensor in the individual's bed.
c) Sensors may by employed to calculate similar, complementary, or different risk metrics. For example, but without limitation, an individual's movement data may be compared to their baseline movement data to calculate a measure of lethargy which may be related to disease.
d) Different sensor technologies may be employed to measure similar biophysical responses to disease.
e) Different biophysical responses to disease may be measured using suitable sensors.
f) Different processing, storage and communications architectures may be used.
g) Different threshold metrics may be used for resting heart rate and resting body temperature.
h) Ambulatory rather than resting metrics may be used.
i) Estimates of resting metrics may be generated from continuous measurements.
j) Different periods to establish any baseline for an individual may be used.
k) The collected alert status and contact lists may be analyzed to produce contact maps and routes of potential disease transfer.
l) Individuals may be alerted after contacts that had no alert when the contact happened, but subsequently developed symptoms leading to an alert.
m) Rather than resetting the alert status when the individual is asymptomatic, the alert status may be maintained for a pre-defined period.
n) Processing of data in any combination (e.g. sensor, alert status, contact information) may occur either locally (“at the edge”) or centrally (e.g. using a cloud-based software system).

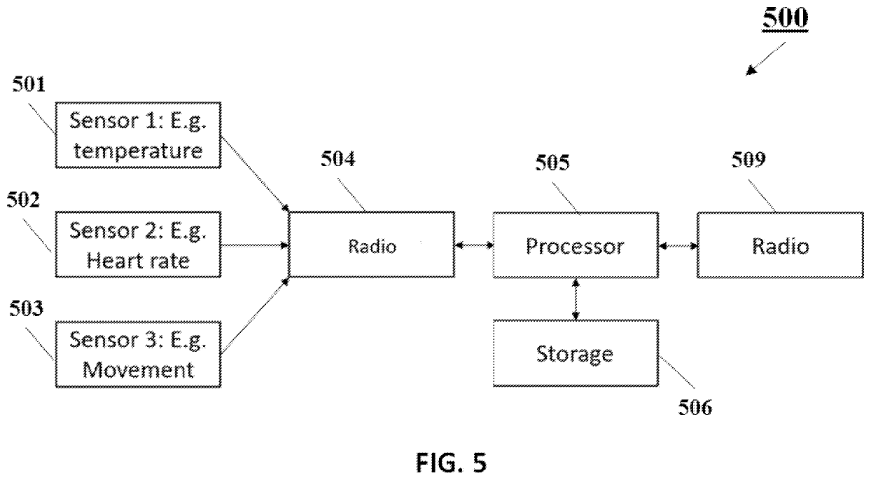

Referring now to FIG. 5, a distributed sensor system 500 is illustrated. In system 500, a temperature sensor 501 may be positioned remote from its human target. For example, this could be a temperature sensor that is mounted on a wall at an airport or a sign on a city street. This remote sensor would be able to, using an infrared technology, assess the temperature of an individual. Then, using face recognition software, or through a connection to the user's smart phone, would be able to associate the temperature with the user. The temperature then to be communicated by radio 504 to a centralized location. In another example, the temperature sensor 501 would be installed at a business for assessing temperatures of employees, and employee identification could be made using facial recognition software or through detections of an employee bad, for example. In a similar way, a movement sensor 503 may be a remotely positioned camera for determining present movement of a human being, and to assess how long they have been in a state of low movement. This would allow a central system to determine if they've been stationary long enough for stable temperature and heart rate measurement. As the heart rate sensor 502, this could be, for example, a smartwatch that a user wears that communicates via a radio to a smart phone. Although radio 504 is illustrated as a single radio, it will be understood that multiple radios may be used. The information from sensor 501, 502, and 503 is passed to processor 504 where it can be analyzed and also stored in storage 506. Processor 505 implements a similar process as described with reference to FIG. 3, and therefore will not be described here. Again, as with the process of FIG. 3, radio 509 may be able to communicate alerts to other radio devices.

While particular preferred and alternative embodiments of the present intention have been disclosed, it will be appreciated that many various modifications and extensions of the above-described technology may be implemented using the teaching of this invention. All such modifications and extensions are intended to be included within the true spirit and scope of the appended claims. The claims identified below do not in any way limit the breadth and scope of the disclosure in the text and drawings provided in this provisional application. Further, the information and detail in the claims below are also considered part of the detailed disclosure of the specification.

What is claimed is:

1. A human-wearable sensor package, comprising:
a temperature sensor;
a heart-rate sensor;
a motion sensor;
a processor performing the steps of:
determining, using the motion sensor, that the human is sufficiently at rest to allow an accurate temperature or heart-rate measurement;
taking a temperature measurement or a heart-rate measurement;
evaluating the temperature measurement or the heart rate measurement to determine that the human wearing the sensor package exceeds pre-defined criteria for having contracted a disease;
setting an alarm level responsive to the determining step; and
forming an alert message;
a radio for transmitting the alert message;
storage; and
a power source.

2. The human-wearable sensor package according to claim 1, wherein the power source is a battery or harvests electricity from motion, light, or an electro-chemical source.

3. The human-wearable sensor package according to claim 1, further including an adhesive pad for coupling the sensors package to human skin.

4. The human-wearable sensor package according to claim 1, further including an environmental sensor.

5. The human-wearable sensor package according to claim 1, further including a position location sensor.

6. A human-wearable sensor package, comprising:
a first physiological sensor;
a second physiological sensor;
a third physiological sensor;
a processor performing the steps of:
determining, using the first, second and third sensors, that the human wearing the sensor package exceeds pre-defined criteria for having contracted a disease;
setting an alarm level responsive to the determining step; and
forming an alert message;
a radio for transmitting the alert message;
storage; and
a power source.

7. The human-wearable sensor package according to claim 6, wherein:
the first physiological sensor is a temperature sensor;
the second physiological sensor is a heart-rate sensor; and
the third physiological sensor is a motion sensor.

8. The human-wearable sensor package according to claim 7, wherein (1) the processor further determines, using data from the motion sensor, that the human is sufficiently at-rest to take a temperature using the temperature sensor, or (2) the processor further determines, using data from the motion sensor, that the human is sufficiently at rest to take a heart rate reading using the heart-rate sensor.

9. The human-wearable sensor package according to claim 6, wherein the power source is a battery or harvests electricity from motion, light, or an electro-chemical source.

10. The human-wearable sensor package according to claim 6, further including an adhesive pad for coupling the sensors package to human skin.

11. The human-wearable sensor package according to claim 6, further including an environmental sensor.

12. The human-wearable sensor package according to claim 6, further including a position location sensor.

13. The human-wearable sensor package according to claim 6, wherein at least one of the physiological sensors is remote and communicates to the human-wearable sensor package using the radio.

14. A method for determining if a human is at risk of being infected, comprising:
providing a human wearable sensor package further comprising:
a first physiological sensor;
a second physiological sensor;
a third physiological sensor;
a processor;
a radio;
storage; and
a power source
determining a baseline range for the first physiological sensor and a baseline range for the second physiological sensor;
using the third physiological sensor to determine if the human is sufficiently at rest that the first physiological sensor and for the second physiological sensor are enabled to take accurate measurements;
taking a first measurement using the first physiological sensor and taking a second measurement using the second physiological sensor;
comparing (1) the first measurement to the first baseline range and (2) the second measurement to the second baseline range;
setting, responsive to the comparing step, an alert level;
generating an alert message according to the alert level; and
transmitting, using the radio, the alert message.

15. The method according to claim 14, wherein if the comparing step results in no alert level being set, then not transmitting any message regarding the first and second measurements.

16. The method according to claim 14, wherein:
the first physiological sensor is a temperature sensor;
the second physiological sensor is a heart-rate sensor; and
the third physiological sensor is a motion sensor.

17. The method according to claim 16, wherein (1) the processor further determines, using data from the motion sensor, that the human is sufficiently at-rest to take a temperature using the temperature sensor, or (2) the processor further determines, using data from the motion sensor, that the human is sufficiently at rest to take a heart rate reading using the heart-rate sensor.

18. The method according to claim 16, wherein the power source is a battery or harvests electricity from motion, light, or an electro-chemical source.

19. The method according to claim 16, further including the step of receiving an alert message on the radio that another near-by user is at risk of being infected and generating a local alert to that effect.

20. The method according to claim 16, further including the step of receiving multiple alert messages on the radio that another near-by user is at risk of being infected, determining how long that user has been nearby, and generating a local alert to that effect.
